(19) United States
(12) Patent Application Publication
(10) Pub. No.: US 2007/0256696 A1
(43) Pub. Date: Nov. 8, 2007

(54) METHOD FOR PRODUCING POLYMERIC SURFACES WITH LOW FRICTION

(75) Inventors: Alisa BUCHMAN, Haifa (IL); Menachem MALEK, Carmiel (IL)

Correspondence Address: MERCHANT & GOULD PC, P.O. BOX 2903, MINNEAPOLIS, MN, 55402-0903, US

(73) Assignee: RAFAEL-ARMAMENT DEVELOPMENT AUTHORITY LTD., Haifa (IL)

(21) Appl. No.: 11/693,444

(22) Filed: Mar. 29, 2007

(30) Foreign Application Priority Data
Apr. 6, 2006 (IL) 174841

(51) Int. Cl. A61L 29/14; A61L 33/00

(52) U.S. Cl. 128/897

(57) ABSTRACT

The invention relates to the use of ceramic, plasma-deposited, layers for manufacturing plastic articles whose function requires low-friction surface. The invention further provides a process of manufacturing a polymer object having a low-friction surface, comprising i) providing a mold defining the shape of said object; ii) coating said mold that defines the shape of said low-friction surface with a ceramic layer, the layer being formed by plasma deposition; and iii) molding the polymer in said mold. The invention also provides a polymer article having a low-friction surface created by molding on a plasma-deposited ceramic surface.

### FIELD OF THE INVENTION

The present invention relates to polymeric articles having a low-friction surface, particularly articles used in electronic or medical systems. The invention further relates to a method of manufacturing polymeric articles whose outer or inner surface has a low friction coefficient.

### BACKGROUND OF THE INVENTION

A low-friction surface is required in numerous applications of polymeric products. An improved sliding, for example, increases wear resistance, and prevents electrostatic charge, and prolongs the lifetime of flat cables used in many electronic assemblies; a low-friction surface of medical devices, such as catheters, tubing and articulating implants, which improves their performance. The friction of a polymeric surface may be lowered by using a lubricant, or by modifying the polymeric material, wherein the modification may occur before or after molding the polymeric product. The modification may comprise additives either admixed to the molding mixture or used as coating for the net-shaped polymeric surface. Said additives usually comprise environmentally harmful materials, such as fluorinated hydrocarbons. U.S. Pat. No. 5,274,049 describes a method for reducing coefficients of friction of elastomeric articles, comprising exposing said articles to gaseous fluorine. U.S. Pat. No. 6,053,171 discloses plasma treated polymeric tubing with modified slip characteristics of the outer surface, achieved by deposition of a monomer during exposure to the plasma glow discharge. Current methods of manufacturing polymeric articles, comprising injection or compression, do not provide surfaces smooth enough without including complicated or environmentally unfriendly techniques. For example, impregnation of surfaces with fluoro-olefins is a complex process, and a dangerous one for the workers. Additives used on medical implants or devices may lower or even prevent biocompatibility. It is therefore an object of this invention to provide a method for manufacturing polymeric articles with required tribological properties without drawbacks of the currently used methods.

This invention aims at providing molded polymeric articles with a low-friction surface, wherein the smoothness is achieved without use of coatings, or additives such as fluorine or fluorinated hydrocarbons. The smoothness provided by the invention is permanent, whereas coatings on plastics are temporary and can easily peel or chip off due to poor adhesion to the plastic article or due to different coefficients of thermal expansion.

This invention further aims at providing molded polymeric articles with a low-friction surface, wherein the smoothness is achieved without need of modifying the surface after molding, which is also cost effective as the production is a direct, one step, production comprising injection with smoothening in one step.

It is another object of this invention to provide articles comprising a polymeric surface with a low friction coefficient, wherein the surface may be an outer or inner surface of the article.

Other objects and advantages of present invention will appear as description proceeds.

### SUMMARY OF THE INVENTION

The present invention provides a process of manufacturing a polymer object having a low-friction surface, comprising i) providing a mold defining the shape of said object; ii) coating said mold that defines the shape of said low-friction surface with a ceramic layer, the layer being formed by plasma deposition; and iii) molding the polymer in said mold. In a preferred method of the invention, said plasma deposition comprises thin layer coating, wherein said ceramic layer comprises a material preferably selected from the group consisting of AlN, Al₂O₃, SiC, CrN, TaN, SiO₂, and Si₃N₄, but other suitable ceramic compositions can be used that smoothly coat. The mold may comprise metal, ceramic, composite, or polymer, and it may have any shape and can by manufactured by any method known in the art. Said polymer may comprise thermoset or thermoplasts, and may comprise filled or unfilled polymers. Said mold is made, for example by a casting or machining method. The mold may be made of materials comprising aluminum, steel, brass, composite materials, and polymeric materials. Said polymer of said object having a low-friction surface may consist of silicon, polyethylene, polypropylene, polycarbonate, polyacetal, and fluoropolymers. In another preferred embodiment of the invention, said low friction surface is an outer surface or a part of an outer surface of an article selected from the group consisting of cables, tubing, and electrical or optical lines. In another preferred embodiment of the invention, said low friction surface is an outer surface or a part of an outer surface of an article selected from catheters, implants, tubing, and device for medical use. In a further preferred embodiment of the invention, said low friction surface is an inner surface of an article selected from catheters, implants, tubing, syringes, and device for medical use. As for the type of molding, it may be injection, compression, extrusion, or calendaring, or other method known in the art. In one preferred embodiment of the invention, said polymer comprises silicon and said molding comprises injection molding.

The invention is directed to the use of a plasma-deposited ceramic surface as a template for the preparation of a low-friction polymer surface. For example, a mold pin may be coated with Al₂O₃ by plasma deposition, and used as a template, wherein a polymer is injected around the pin as a tube, resulting in a tube exhibiting highly smooth inner surface.

The invention provides a polymer article having a low-friction surface created by molding on a plasma-deposited ceramic surface. Said article exhibits low friction wear, and preferably low static electricity. Said article may have any shape manufactured by known methods, such as the shape of sheets, cables, fibers, tubes, etc. In one embodiment, the article of the invention is selected from the group consisting of accessories of electronic systems, accessories of optical systems, and medical accessories. Examples are electrical or optical coated lines. In a preferred embodiment of the invention, the article having a smooth polymer outer surface is a flat flexible cable. An example of a medical application taking advantage of the invention is using a syringe with smooth inner surface for the injection of lenses applied during eye surgery, when operating short or long sighted patients.

### DETAILED DESCRIPTION OF THE INVENTION

It has now been found that polymeric articles obtained by injection or compression molding, or by extrusion or calendaring, in a mold provided with a plasma-deposited ceramic surface have surprisingly smooth surfaces. The friction coefficient of the articles attained by the invention, is lower than provided by the prior art methods. The surfaces prepared according to the invention may have a friction coefficient of from 0.1 to 0.3 For example, in one comparison experiment, as shown in Examples below, values of vertical/horizontal friction coefficients have been 0.3/0.2 for the method of the invention, whereas fluoro-coating has provided 0.4/0.3, and classical injection molding 0.5/0.3. The invention enables manufacturing products having low surface friction, which products have turned out to exhibit additional advantageous features, including low friction wear, and low static electricity. The method according to the invention may be utilized, for example, for manufacturing flat cables, medical tubing or catheters or implants, etc.

Plasma deposition of inorganic layers provides surfaces with low friction coefficients. For example, EP 415 851 describes a ceramic coating created by plasma deposition for use in bearings. In the present invention, a smooth ceramic surface is utilized as a master substrate to be duplicated on a plastic surface. Plasma-deposited ceramic film or layer is used in a novel way, obviating the need of lubrication, replacing the previous methods for modifying the plastic surfaces, and providing self-lubricated surfaces in a simple, cost-effective and environmentally safer technique, wherein the technique provides products exhibiting additional benign features, such as low static electricity, better biocompatibility, and high working stability.

The method of the invention comprises preparing a mold with a thin ceramic layer, wherein the layer is formed by plasma-assisted deposition, using methods known in the art, comprising for example ion-beam assisted deposition, ion beam deposition, plasma-assisted chemical vapor deposition, plasma-sputter deposition, diamond like carbon films deposition. The ceramic surface has been found to be highly wear-resistant when used in methods according to the invention, and enduring thermal and mechanical burdens related to it uses. The ceramic surface in the mold used in this invention should preferably have a friction coefficient lower than 0.05. A plasma deposition method is exploited for manufacturing a coated mold with high abrasion resistance, and long lifetime. The mold may be made of materials comprising metals composites, ceramics or plastics, and may be produced, e.g. by casting or machining and then coated by plasma-deposition, the thickness of the layer being, for example, about 1-5 μ. The mold may comprise other substrates known in the art of molding polymers, wherein said substrate is first chemically cleaned to remove contaminants, then it is placed in a vacuum chamber followed by evacuating the chamber; the substrate may be bombarded with a plasma of energetic ions to remove residual interfering contaminations and to activate the surface, and finally a smooth coating is deposited by plasma deposition. Preferred ceramic coatings comprise Al₂O₃, Si₃N₄, SiC, CrN, TaN or AlN. The deposition process is continued until attaining the desired thickness of the coating layer, which is usually in the range of 0.5-50 μm, preferably from about 1 to about 20 μm. The coating is very stable for prolonged use of the mold in processing polymers. For example, a coating used in the method of invention underwent in one test over 10,000 injections without wear. The invention utilizes molds with deposited ceramic coatings for shaping polymeric articles, wherein the known techniques of injection, compression molding or extrusion may be utilized. For example, polymer powder or pellets are poured into the injector machine, the spiral rod causes the resin to melt by converting mechanical energy into heat, and the liquid resin is injected into the preheated mold to produce a net shaped article.

Thus, the invention provides a method of producing a plastic molded object, comprising preparing a mold assembly including a cavity comprising a plasma-deposited ceramic coating, which cavity imparts the shape to the surface intended to have a low friction coefficient. The article of the invention may be made of various polymers, comprising thermoplastic and thermosetting resins. In many preferred applications, silicon is a polymer of choice. The prior art techniques process polymers, such as silicon, to be smooth by coating its surface, but there are numerous drawbacks, one of them being instability of the smooth layer, which may peel off as the coefficients of thermal expansion are different, and cracks may occur while drawing, heating, or cooling.

The method of the invention comprises preparing a mold of which inner surface is covered by plasma deposited ceramic layer, molding a polymer to obtain an article having the desired shape and a surface exhibiting a low friction coefficient. Said molding may comprise the steps of heating the mold assembly to a temperature lower than a softening temperature of a molten resin, filling the cavity with the molten polymer, cooling the molten resin to a temperature lower than the softening temperature of the resin, resulting in an article with smooth surface.

Among important applications of frictionless polymer surfaces is the manufacturing flat flexible silicon cables, needed in many electronic systems. Flexible flat cables, consisting of many parallel lines held together by a polymer, may touch each other during their service life, and the low friction is an important prerequisite of the functional system, otherwise, wear and static electricity may damage the system. Silicon is often used for binding the parallel lines, and their surface is coated by fluoro-olefins to lower the friction, or various components are added to the polymer; some additives are intended to lower the friction, other to prevent the separation of coating layers. The prior art, thus, solves the problem by adding materials into/onto the polymers to lower the friction, wherein the techniques involve multiple steps, complicated from the technological viewpoint, and from the viewpoint of environment safety. The invention now provides a method for manufacturing the flat cables in one integrated and safe process, without need of the additives, by casting silicon into a suitable mold and by obtaining a form that enables embedding the lines into a flat cable. The frictionless surface of the cables is obtained by creating a smooth ceramic layer in the mold by means of plasma deposition, and by subsequent molding a plastic in said mold and copying the perfectly smooth surface of the ceramic layer onto the surface of the cast polymer, thereby forming a plastic article having a low friction surface. The friction coefficient of the cables obtained by the method of the invention is lower than in case of cables obtained by the prior art methods using shiny polished molds. The mold with ceramic surface is stable and can be used repeatedly without wear and loss of efficiency. The process is simple, safe, reproducible, and provides molds usable for very long periods of time. The process of the invention can replace the known, less efficient or more expensive, or environmentally dangerous techniques.

There are numerous other applications of the method of the invention, such as producing medical devices and articulating implants. For example, there are needs for syringes with smooth inner surface; the invention provides a method for manufacturing such syringes by preparing a mold with perfectly smooth ceramic surface by plasma deposition, and by copying the surface by the inner syringe surface injected to the mold. Such syringe will comply, for example, with the need of injecting lenses into the eye during operations. The absence of potentially harmful components in the polymer is especially important in the medical applications, as the coated surface is stable and biocompatible. For example, plasma deposition of Al₂O₃ may be carried out onto the surface of molds prepared for polyimide implants of an acetabular cup, comprising compression molding of polyimide powder at high pressure and temperature in the coated mold, thereby obtaining articulating implants with high surface smoothness and wear resistance.

The invention thus relates to the use of ceramic, plasma-deposited, layers for manufacturing plastic articles whose function requires low-friction surface. In a preferred embodiment of the present invention, articles having a surface at least partially covered with a polymer are provided for applications in which low-friction surface is advantageous or necessary. In a further aspect, the invention is directed to providing molds for casting polymeric articles, which molds have inner surface covered with a plasma-deposited ceramic film or coat of suitable dimensions.

The invention will be further described and illustrated in the following examples.

### EXAMPLES

### Materials and Methods

Silicon was purchased in General Electric Co. as a 2-component liquid (resin+accelerator). Si, Al, Ti, Cr, of the 99.99% purity grade were purchased in Good Fellow to serve as a source of the plasma deposition. The gases, argon, oxygen, nitrogen, were purchased in Oxygen Industry, Haifa.

Plasma depositing was carried out using a hemi-spherical vacuum chamber, 60 cm diameter, with a sputtering apparatus, into which the mold was placed. Vacuum chamber was evacuated by means of diffusion pump 1-5×10⁻³ mbar. High voltage (1-4 kV) and low current (0.5-5 mA) were generated, combined with magnetic field (10-30 kA/m). The chamber was purged with active gas diluted with argon in order to clean the surface of the mold by mild etching. The metal source generates high energy particles which, combined with the inert gas, are sputtered onto the mold, coating it with a thin film. Film thickness is determined by the sputtering time. Metal molds for shaping model flat silicon cables were made by machining aluminum block, sputtered with Al₂O₃ layer. Model cables for comparing the friction coefficients were prepared from 2-component silicon resin injected into the mold containing the wires. The friction coefficients, vertical and horizontal, were checked according to the method of pin on disc where the pin is stainless steel and the disc is the silicone sample.

### Example 1

An aluminum mold for shaping flat cables machined from a block was placed in the high vacuum chamber of the sputtering apparatus. The air was evacuated to a pressure of about 10-3 or less, and high voltage (1-4 KV) and low current (0.5-5 mAmp) was generated and combined with magnetic field of 10-30 KA/m. The surface of the mold was bombarded with high energy particles to remove any residual contamination. After sputter-etching the surface, precursor gases containing O₂ diluted in Ar are introduced into the chamber containing an Al source producing plasma. The ions strike the surface of the mold with high energies. The thickness of the deposited layer was from 0.1 to 100 μm, when estimated by time of deposition. It was found that a layer of from about 0.5 to about 20 μm was usually sufficient for stable repeated use of the mold.

In three experiments, the following coatings were prepared: Si₃N₄, about 0.5 μm thick; AlN about 0.4 μm thick; and Al₂O₃ about 0.5 μm thick. A mixture of two part silicon resin was injected into the pretreated mold, at room temperature. Three samples (S1, S2, S3) of flat cables were thus obtained.

### Example 2

A sample of flat cables of the same dimensions as those obtained in Example 1, but prepared in a mold not coated with a plasma-deposited ceramic layer, was provided, and its surface was processed by fluorinated coating, according a proprietary method. This comparative sample (S4) was obtained from CiCoil and NeuBuck Corp. in USA.

### Example 3

A sample of flat cables of the same dimensions as those obtained in Example 1 was prepared by injecting to a polished aluminum mold. A comparative sample (S5) was obtained.

### Example 4

Samples S3 to S5 of flat cables prepared according to Examples 1 to 3 were characterized visually and under pin-on-disc friction test. The following table shows the results, demonstrating that the novel method according to the invention provides smoother surfaces. Further characterizations showed that, in addition to superior tribological properties, the articles show additional advantages, such as low static electricity, and low wear.

TABLE 1
Friction coefficients of three samples, S4 and S5 prepared
according to prior art techniques, as described in Examples 2
and 3, and S3 prepared according to the invention, as described
in Example 1.

| | Vertical/horizontal |
|---|---|
| Sample type | friction coefficient |
| Fluoro-coated (S4) | 0.3/0.4 |
| Injection (S5) | 0.3/0.5 |
| Plasma-coated (S3) | 0.2/0.3 |

### Example 5

Using the method of the invention, a steel syringe was prepared, whose outer surface was coated with ceramic thin layer deposition which exhibited very low friction coefficient. A mold for shaping the outer plastic film of the syringe was made by plasma deposition, similarly as described in Example 1. Polypropylene molten resin was prepared, and injected into the space between the mold and the steel syringe, followed by cooling.

The syringe was evaluated by preparing polymer flexible lenses of the type used in eye surgery. The comparison with other syringes showed that using the syringes prepared with the plasma deposited mold enabled better and smoother passage of the lens inside the syringe without damage.

While this invention has been described in terms of some specific examples, many modifications and variations are possible. It is therefore understood that within the scope of the appended claims, the invention may be realized otherwise than as specifically described.

1. A process of manufacturing a polymer object having a low-friction surface, comprising
i) providing a mold defining the shape of said object;
ii) coating said mold that defines the shape of said low-friction surface object with a ceramic layer, the layer being formed by plasma deposition; and
iii) molding the polymer in said mold.

2. A process according to claim 1, wherein said plasma deposition comprises thin layer coating.

3. A process according to claim 1, wherein said ceramic layer comprises a material selected from the group consisting of AlN, Al₂O₃, SiC, CrN, TaN, SiO₂, and Si₃N₄.

4. A process according to claim 1, wherein said mold is made by a casting or machining method, and comprises materials selected from aluminum, steel, brass, composite material, and plastic material.

5. A process according to claim 1, wherein said low friction surface is an outer surface or a part of an outer surface of an article selected from the group consisting of cables, tubing, and electrical or optical lines.

6. A process according to claim 1, wherein said low friction surface is an outer surface or a part of an outer surface of an article selected from catheters, implants, tubing, and device for medical use.

7. A process according to claim 1, wherein said low friction surface is an inner surface of an article selected from catheters, implants, tubing, syringes, and device for medical use.

8. A process according to claim 1, wherein the polymer of said object having a low-friction surface is selected from the group consisting of thermoplasts and thermosets.

9. A process according to claim 1, wherein the polymer of said object having a low-friction surface is selected from the group consisting of silicon, polyethylene, polypropylene, polyacetal, and fluoropolymers.

10. A process according to claim 1, wherein said molding is selected from the group consisting of injection, compression, extrusion, and calendaring.

11. A process according to claim 1, wherein said polymer comprises silicon and said molding comprises injection molding.

12. A process comprising using a plasma-deposited ceramic surface as a template for the preparation of a low-friction polymer surface.

13. A polymer article having a low-friction surface created by molding on a plasma-deposited ceramic surface.

14. An article according to claim 13, further exhibiting low friction wear.

15. An article according to claim 13, further exhibiting low static electricity.

16. An article according to claim 13, selected from the group consisting of accessories of electronic systems, accessories of optical systems, and medical accessories.

17. An article according to claim 16, being flat flexible cable.

18. An article according to claim 16, selected from the group consisting of catheters, implants, tubing, and syringes.
